(19) United States
(12) Patent Application Publication
(10) Pub. No.: US 2011/0042233 A1
(43) Pub. Date: Feb. 24, 2011

(54) PERMEABLE ELECTROCHEMICAL REACTIVE BIOBARRIER AND METHOD FOR USING THE SAME

(76) Inventors: Yung-Fu Wang, Kaohsiung City (TW); Wei-Ji Chen, Tainan City (TW); Hsu-Wen Hung, Tainan City (TW); Po-Tseng Pan, Pingtung City (TW); Sheng-Shung Cheng, Tainan City (TW)

Correspondence Address: Muncy, Geissler, Olds & Lowe, PLLC, 4000 Legato Road, Suite 310, FAIRFAX, VA, 22033, US

(21) Appl. No.: 12/860,169

(22) Filed: Aug. 20, 2010

(30) Foreign Application Priority Data
Aug. 21, 2009 (TW) 098128130
Jun. 4, 2010 (CN) 201010198686.1
Aug. 19, 2010 (TW) 099127815

(51) Int. Cl. A61L 2/03

(52) U.S. Cl. 205/701; 204/252

(57) ABSTRACT

A permeable electrochemical reactive biobarrier used to prohibit the diffusion of groundwater contaminated by organic compounds is disclosed. The permeable electrochemical reactive biobarrier includes at least a conductive fiber layer and at least a cathode. The conductive fiber layer is applied with a proper voltage, and the cathode is disposed at one side of the conductive fiber layer. Herein, the conductive fiber layer can be used as the electron acceptor for respiration occurred by local soil microorganisms, which grow on the surface of the conductive fiber layer. Accordingly, the biodegradation of organic compounds can be continued without additional oxidants.

### CROSS REFERENCE TO RELATED APPLICATIONS

This Non-provisional application claims priority under 35 U.S.C. §119(a) on Patent Application No(s). 098128130 and 099127815 filed in Taiwan, Republic of China on Aug. 21, 2009 and Aug. 19, 2010, the entire contents of which are hereby incorporated by reference.

### BACKGROUND OF THE INVENTION

1. Field of Invention

The present invention relates to a permeable electrochemical reactive biobarrier and, in particular, to a permeable electrochemical reactive biobarrier capable of degrading organic contaminants efficiently without adding additional chemical agents for further preventing groundwater pollution.

2. Related Art

The term of “reactive biobarrier” is one of the means to prevent contaminants distributing with the groundwater. It is established at the gas station, oil refineries, or even at some petrochemical plants to prevent the spread of groundwater polluted by organic contaminants. Practically, the reactive biobarrier is disposed perpendicularly at the downstream of polluted groundwater.

Conventional reactive biobarrier is water permeable. The principle is that when groundwater flows through the permeable reactive biobarrier, the contaminants in water are blocked by the permeable reactive biobarrier and then removed by physical methods (e.g. precipitation), chemical methods (e.g. oxidation, or reduction) or biological methods (e.g. biodegradation) to prevent the spread of polluted groundwater.

However, most physical methods exists a problem of ineffectiveness. Because the features of contaminants are not exactly the same, various contaminants cannot be removed by one physical method. Furthermore, it costs manpower and resources to remove remaining contaminants. If applying chemical methods to degrade organic contaminants, the problem of depletion and replacement of reaction agents must be considered. If applying biological degradation mechanism, how to provide sufficient electron acceptors is an existing issue. It requires a large amount of energy to supply the system and, in the meanwhile, results in uneven mass transfer easily. Accordingly, it causes the problems of replenishing or adding additional oxidants (as electron donors for microorganisms) resulting in the increase of remediation cost.

### SUMMARY OF THE INVENTION

To solve the existing issue of the conventional reactive biobarrier, a novel biobarrier is disclosed after several research and thoughts by the inventors. In detailed, the biobarrier includes a conductive fiber with large surface area and good permeability. Additionally, the relevant degrading microorganisms of the biobarrier are formed by the direct attachment and growth of local existing soil microorganisms. The biobarrier is capable of continuously degrading organic contaminants by applying a proper voltage without adding additional microorganisms and general oxidants.

An objective of the present invention is to provide a permeable electrochemical reactive biobarrier capable of degrading organic contaminants efficiently without adding additional chemical agents for preventing groundwater pollution.

To achieve the above-mentioned objective, the permeable electrochemical reactive biobarrier of the present invention includes at least a conductive fiber layer (i.e. biological anode) and a cathode. The conductive fiber layer is applied with a proper voltage, and the cathode is disposed at one side of the conductive fiber layer. Herein, the cathode is used to balance the total electronic charges.

Preferably, the conductive fiber layer is formed by a conductive carbon fiber, a metal fiber or their combination. More preferably, the conductive carbon fiber is preferably a conductive activated carbon fiber.

The present invention also discloses a method for operating a permeable electrochemical reactive biobarrier including the steps of providing the permeable electrochemical reactive biobarrier including a conductive fiber layer and a cathode; and applying a proper voltage to the permeable electrochemical reactive biobarrier. Herein, the cathode is used to balance the total electronic charges.

Preferably, the conductive fiber layer is formed by a conductive carbon fiber, a metal fiber, or their combination. More preferably, the conductive carbon fiber is preferably a conductive activated carbon fiber.

The present invention provides the permeable electrochemical reactive biobarrier for local soil microorganisms to attach and growth spontaneously. Practically, the biobarrier is capable of degrading organic contaminants effectively by just applying a proper voltage as the electron acceptors of the microorganisms instead of the conventional method of adding additional chemical oxidants. It reduces the remediation cost and provides the excellent prevention effect of groundwater pollution.

### BRIEF DESCRIPTION OF THE DRAWINGS

The invention will become more fully understood from the detailed description and accompanying drawings, which are given for illustration only, and thus are not limitative of the present invention, and wherein:

FIG. 1 is a schematic figure of a permeable electrochemical reactive biobarrier according to an embodiment of the present invention;

FIG. 2 is a top view of the permeable electrochemical reactive biobarrier of FIG. 1 that is in operation;

FIG. 3 is a partial enlarged figure of the permeable electrochemical reactive biobarrier shown in FIGS. 2; and

FIG. 4 is a figure indicating the correlation of benzene concentration and biological current in the example of the present invention.

### DETAILED DESCRIPTION OF THE INVENTION

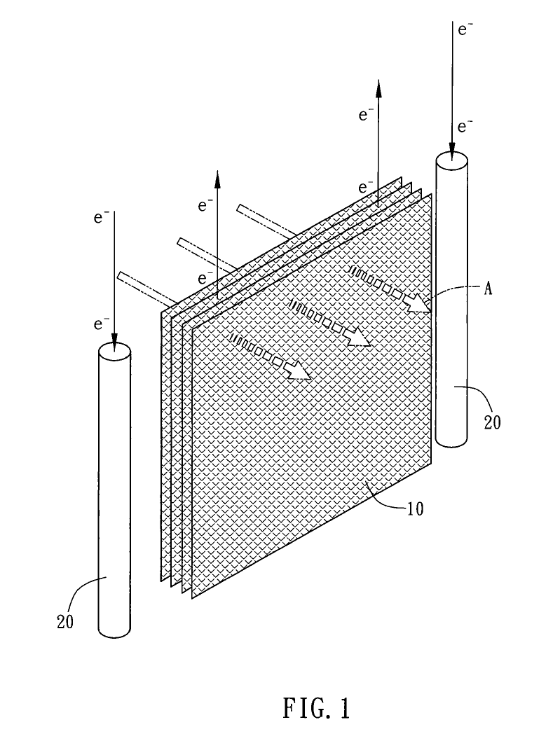

As shown in FIG. 1, a permeable electrochemical reactive biobarrier of the present invention includes at least a conductive fiber layer, which is applied with a proper voltage, and at least a cathode. The material of the conductive fiber layer is not limited and can be any material with electrical conductivity, and it is preferably a conductive carbon fiber, a metal fiber or their combination. In the present embodiment, the conductive carbon fiber is a conductive activated carbon fiber, and the conductive fiber layer is a conductive activated carbon fiber layer 10 for example. As shown in FIG. 1, the permeable electrochemical reactive biobarrier includes a conductive activated carbon fiber layer 10 and a cathode 20. The thickness of the conductive activated carbon fiber layer 10 is preferably less than or equal to 15 cm. The material of the cathode 20 is a rod-shaped or net-shaped object made of other metal and disposed adjacent to the conductive activated carbon fiber layer 10. The disposition distance between the conductive activated carbon fiber layer 10 (i.e. anode) and the cathode is less than or equal to 20 meters, and is preferably less than or equal to 1 meter to prevent the short circuit by the direct contact of the two electrodes. For example, if the conductive activated carbon fiber layer 10 (anode) and the cathode are both disposed in the ocean that has great electrical conductivity, the distance therebetween can be increased up to 20 meters.

The conductive activated carbon fiber layer 10 is disposed perpendicularly in the downstream of the polluted groundwater. As shown in FIG. 1, the reference numeral A shows the flow direction of the groundwater. The cathode 20 is disposed adjacent to the lateral sides of the conductive activated carbon fiber layer 10. Relevant contaminant degrading bacteria can spontaneously attach to and then grow on the surface of the conductive activated carbon fiber layer 10. The electrons generated from the biochemical reaction of the contaminant degrading bacteria can be obtained for the normal operation of the reactive biobarrier by applying a proper voltage, such as from −10 to 10 volts and, preferably from −0.5 to 0.5 volts (versus an Ag/AgCl reference electrodes), without adding additional oxidants. However, if the applied voltage is improper, the electrons inside the microorganisms will be overly captured, which may result in the death of the microorganisms and the decreasing rate of contaminant degradation.

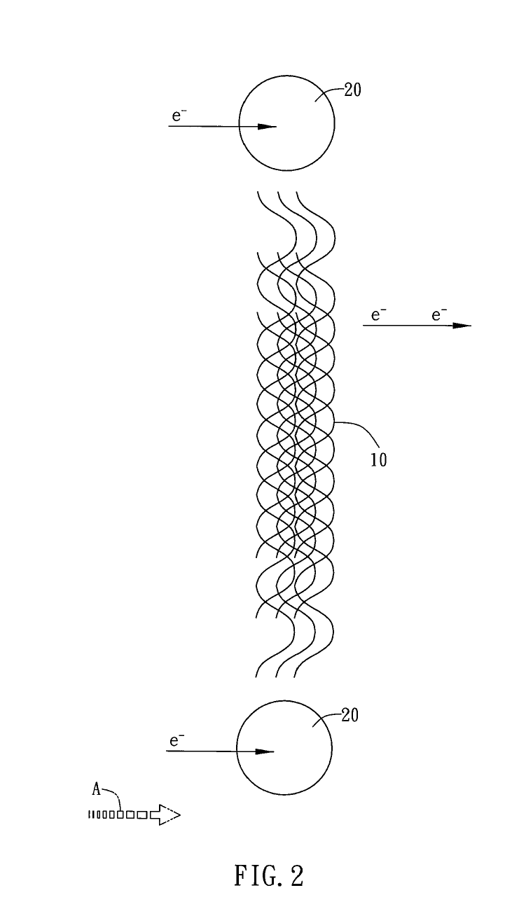

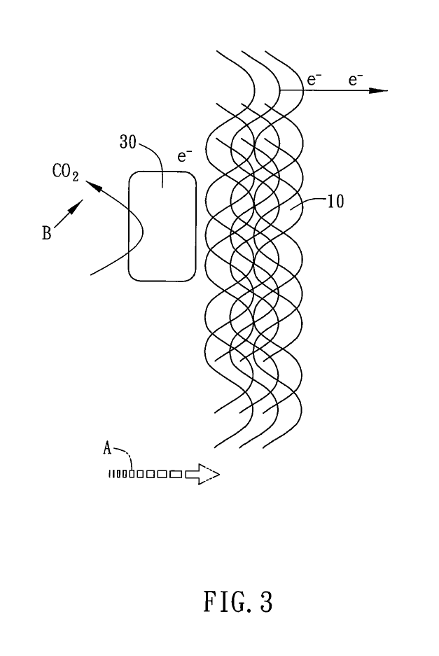

As also shown in FIGS. 2 and 3, since the contaminant degrading bacteria of the electrodes is from existing microorganisms 30 in soil, while the electrodes are applied with a proper voltage, the electrons generated from the microorganisms 30 in the degrading process of organics are continuously accepted at the anode. Afterward the electrons are conducted to the cathode via an external circuit and then induce reduction reaction such as the generation of H₂ on the surface of the cathode for balancing entire electric charge. While the polluted groundwater flows through the conductive activated carbon fiber layer 10 (reference numeral B indicates the direction of the chemical reaction of the organic contaminants), the microorganisms 30 degrade effectively the organic contaminants and then generate carbon dioxide (CO₂) without adding additional oxidants.

### EXAMPLES

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

### Example 1

### Test of Degradation Efficacy of Local Microorganisms (Using the Electrode as an Electron Acceptor)

In the present example, benzene was used as a target organic contaminant (its concentration is about 350 ppm), and microorganism complex polluting soil was used as a seeding source. The test of degradation efficacy and biological current was performed in a reaction vessel. After applying with the voltage of 0.2 volts (versus an Ag/AgCl reference electrodes), it is obtained that the surface of the conductive activated carbon fiber is gradually covered by a biofilm.

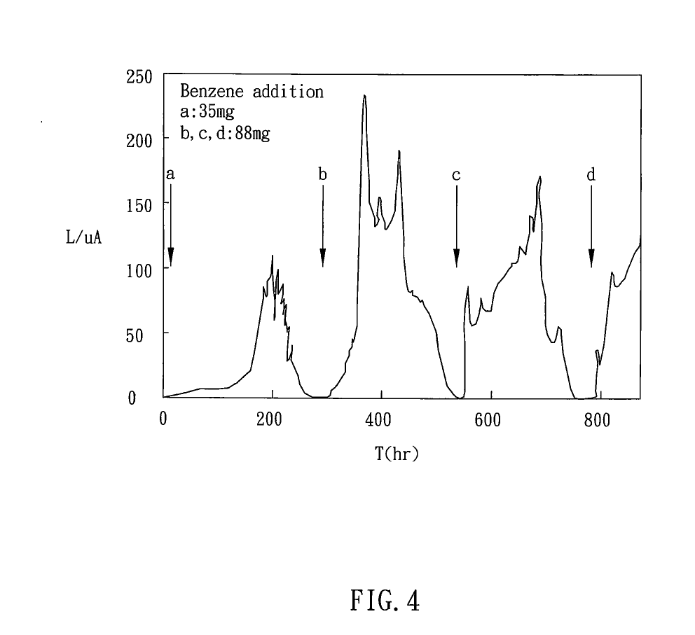

FIG. 4 shows the correlation of benzene existent and biological current. In the beginning of the experiment, 140 ppm benzene was added (shown as the arrow a in FIG. 4) and no current was detected (indicating that benzene is not oxidized directly by the electrode). Afterward the current is gradually rising, and the maximum was up to 100 μA. The current was eventually down to 0 μA after 300 hr. However, when benzene was added again (shown as the arrows b, c and d (the individual concentration is about 350 ppm)), the current generated from the reaction was detected significantly and the frequency of current generation was gradually increased. After the experiment, the microorganisms of the biofilm on the electrode were analyzed. The results of the relevant molecular biological analysis indicated that some microorganisms generating electrons had been cultivated in considerable quantities. In addition, the evidences proved that benzene was oxidized to generate the current by the microorganisms instead of the electrode directly.

In accordance with the aforementioned experiment, the reactive barrier of the present invention was expected to adapt to the condition which the benzene concentration suddenly increased about 350 ppm. It indicated the flexibility of the present invention in application.

### Example 2

### Stimulation Test in Soil Column for Conforming Permeable Electrochemical Reactive Biobarrier

Example 2 was a test in soil column for stimulating the permeable electrochemical reactive barrier. In the present example, the height of the soil column was 45 cm, and the inner diameter of that was 3 cm. The soil column includes a conductive activated carbon fiber layer (15 cm in thickness) and a platinum (Pt) cathode disposed adjacent to the lateral sides of the conductive activated carbon fiber layer. The distance between the conductive activated carbon fiber layer and the platinum cathode was 5 cm. The test was conducted with continuous flow and hydraulic retention time thereof was within 2 days. The inflow contains the simulated contaminant such as benzene, toluene, ethybenzene and xylene (referred to briefly as BTEX), and its testing concentration was selected from about 19524, 15383, 14981 and 7257 ppb. Both of the inflow and outflow were detected for the remaining amount of BTEX. The result that more than 99% of BTEX was removed indicated the outstanding efficacy of the reactive biobarrier.

In accordance with the aforementioned two examples, the outstanding efficacy of the microorganism reactive biobarrier of the present invention has been conformed. Therefore, when the reactive biobarrier of the present invention is disposed at the downstream of groundwater polluted by organics, it can become a permeable electrochemical microorganism reactive barrier by just applying with a proper voltage (lower than 10 volt versus an Ag/AgCl reference electrodes) for the spontaneous attachment and growth of local soil microorganisms. Therefore, the conductive activated carbon fiber layer of the present invention can degrade organic contaminants effectively by just applying with a proper voltage instead of by way of adding additional chemical agents.

What is claimed is:

1. A permeable electrochemical reactive biobarrier, comprising:
at least a conductive fiber layer applied with a proper voltage; and
at least a cathode disposed at a side of the conductive fiber layer.

2. The permeable electrochemical reactive biobarrier of claim 1, wherein the conductive fiber layer is formed by a conductive carbon fiber, a metal fiber or their combination.

3. The permeable electrochemical reactive biobarrier of claim 2, wherein the conductive carbon fiber is a conductive activated carbon fiber.

4. A method for operating a permeable electrochemical reactive biobarrier, comprising the steps of:
providing the permeable electrochemical reactive biobarrier including at least a conductive fiber layer and at least a cathode, wherein the cathode is disposed at a side of the conductive fiber layer; and
applying a proper voltage to the permeable electrochemical reactive biobarrier.

5. The method of claim 4, wherein the conductive fiber layer is formed by a conductive carbon fiber, a metal fiber or their combination.

6. The method of claim 5, wherein the conductive carbon fiber is a conductive activated carbon fiber.
